(19) United States
(12) Patent Application Publication
(10) Pub. No.: US 2026/0358333 A1
(43) Pub. Date: Nov. 12, 2026

(54) DIP-IN-WELL SENSOR FOR ELECTROCHEMICAL MEASUREMENT AND METHOD OF MANUFACTURING DIP-IN-WELL SENSOR

(71) Applicant: HEXAGONFAB LIMITED, Cambridge (GB)

(72) Inventors: Paolo ROMELE, Cambridge (GB); Benjamin WHITELEY, Cambridge (GB); Edward CATLEY, Leighton Buzzard (GB); Ruizhi WANG, Cambridge (GB)

(21) Appl. No.: 19/515,148

(22) PCT Filed: Aug. 29, 2024
(86) PCT No.: PCT/EP2024/074194

§ 371 Date: Feb. 27, 2026

(30) Foreign Application Priority Data
Sep. 1, 2023 (DE) 10 2023 123 620.2
Nov. 29, 2023 (DE) 10 2023 133 434.4
Jan. 23, 2024 (DE) 10 2024 101 875.5

(51) Int. Cl. G01N 33/543; G01N 27/27; G01N 27/28; G01N 27/327; G01N 33/557

(52) U.S. Cl. CPC G01N 33/5438; G01N 27/27; G01N 27/283; G01N 27/327; G01N 33/557

(57) ABSTRACT

A dip-in-well sensor includes multiple electrodes. The dip-in-well sensor is designed to be submerged into an assay at its distal end (191). The dip-in-well sensor can be connected to a measurement system for performing an electrochemical measurement.

### TECHNICAL FIELD

Various examples of the disclosure pertain to a dip-in-well sensor for electrochemical measurements and a method of manufacturing such dip-in-well sensor. Various examples of the disclosure relate to a measurement system for reading out a dip-in-well sensor and for performing electrochemical measurements based on signals from the dip-in-well sensor.

### BACKGROUND

Electrochemical measurements can be used in various applications. For example, reduction-oxidation electrochemical measurements can be used to investigate biological or chemical assays.

For performing the reduction-oxidization electrochemical measurements, an electrode and a counter-electrode are submerged into a fluid. Then, a potential difference or a current between the electrode and the counter-electrode are measured. Sometimes, one of the electrodes is bio-functionalized to measure certain properties of the assay.

Dip-in-well sensors including the electrodes are used. It is typically required to exchange the sensors from time to time, e.g., after each measurement if the surface of the electrode reacts with the assay, e.g., by molecules binding to the bio-functionalized surface. Accordingly, exchangeable sensors are required.

### SUMMARY

A need exists for advanced dip-in-well sensors and associated measurement systems for electrochemical measurements. In particular, a need exists for sensors that can be bio functionalized, are easy-to-manufacture, and can be conveniently attached to mounting bracket of the measurement system.

This need is met by the features of the independent claims. The features of the dependent claims define embodiments.

A dip-in-well sensor (hereinafter also simply sensor) for electrochemical measurements is disclosed. The sensor can be at least partially submerged in a fluid/assay. The sensor is for being dipped into an assay at its distal end.

Various types of electrochemical measurements can be facilitated including, but not limited to reduction-oxidization electrochemical measurements.

The sensor includes a base. The base may have an elongated shape.

The base has a first surface and a second surface and a circumference.

Without restriction of generality, the “first surface” will now be referred to as “top surface” and the “second surface” will now be referred to as “bottom surface”. This is for purposes of readability.

The sensor also includes at least one metal electrode. The metal electrode is arranged on the top surface of the base. Alternatively, the metal electrode is arranged on the bottom surface of the base. The metal electrode extends between the distal end and a proximal end of the sensor. Thus, the metal electrode is designed to be dipped into the assay, to thereby serve as the electrode or counter-electrode of the electrochemical measurement.

The sensor also includes a substrate slab, e.g., made of a semiconductor such as silicon. One option is that the substrate slab is made of boron-silicate. Other options include glass, ceramic, or polymers. The substrate slab can have an elongated shape. The substrate slab is attached, at its second (bottom) surface, to the top surface of the base. Adhesive attachment can be used. The slab extends along the at least one metal electrode between the distal end and the proximal end.

A further metal electrode is arranged on the first (top) surface of the substrate slab.

Also, the further metal electrode extends between the proximal end and the distal end. The further metal electrode can serve as the electrode or counter-electrode of the electrochemical measurement.

Accordingly, the dip-in-well sensor can be a multi-part system. A first part is implemented by the base that carries the at least one metal electrode. Attached thereto is the second part, i.e., the substrate slab that carries the further metal electrode.

Such a multi-part implementation of the sensor has certain advantages. In particular, separate manufacturing steps can be implemented for the at least one metal electrode attached to the base on the one hand and the further metal electrode attached to the substrate slab on the other hand. Thus, sensitive manufacturing steps such as biofunctionalization of a distal end of the at least one electrode or the further electrode can be carried out without or with reduced interference with other manufacturing steps of the respective other part.

More generally speaking, the at least one metal electrode and the further metal electrode can be separately formed/manufactured on the base and the substrate slab, respectively; then, after forming the electrodes, the substrate slab (can also be termed substrate plate) can be attached to the base.

According to examples, a sensing region of the further metal electrode that is arranged at the distal end of the sensor is bio-functionalized. For instance, such bio-functionalization can pertain to certain molecules being attached to a metal surface of the further metal electrode. These molecules can act as binding receptors for testing chemical coupling to molecules in the assay.

The further metal electrode also forms a connection region that extends away from the sensing region towards the proximal end of the sensor. The connection region can have a smaller width than the sensing region.

The width of the sensing region and the connection region of the further metal electrode is defined in the plane of the respective metal layer perpendicular to the longitudinal direction of sensor extending between the distal end and the proximal end.

In other words, the connection region can form a constriction of the further metal electrode. The further metal electrode includes a narrow center part. This has the effect that the surface of the further metal electrode facing the surrounding- and, accordingly, potentially in contact with the assay-is reduced in the connection region if compared to a scenario in which the connection region has the same width as the sensing region. Thereby, the electrochemical measurement is less sensitive to varying cover ratios of the assay on the connection region. Thus, the signal of the electrochemical measurement predominantly stems from the sensing region of the further metal electrode. Thus, a more reproducible signal is provided, and the electrochemical measurement is more accurate.

The substrate slab can protrude from the top surface of the base and from the at least one metal electrode. Accordingly, in other words, the top surface of the substrate slab can be offset perpendicular to the plane of the further electrode from the top surface of the base. In other words, the top surface of the overall sensor has a topography/profile. Such topography of the sensor perpendicular to the plane of the at least one electrode on the base and the further electrode arranged on the substrate slab can help to align the sensor in a mounting bracket of a measurement system.

According to examples, the at least one metal electrode includes a first metal electrode and the second metal electrode. The first metal electrode and the second metal electrode can be symmetrically arranged with respect to a center axis that is parallel to or coincident with a center axis of the further metal electrode.

According to certain examples, the at least one metal electrode (arranged on the top surface of the base) includes a first metal electrode and a second metal electrode. The substrate slab can then be arranged in-between the first metal electrode and the second metal electrode. A symmetric arrangement-providing mirror symmetry along with the longitudinal center axis of the sensor-can be implemented.

The base can include a narrow dipping region and a wide connection region. The narrow dipping region is for being dipped into the assay and is arranged at the distal end of the sensor; while the wide connection region is for enabling an electrical contact between the at least one metal electrode and a measurement system, as well as between the further metal electrode and the measurement system.

The at least one metal electrode can include a connection region. The connection region is then arranged in the wide connection region of the base and has a width (perpendicular to the longitudinal direction of the at least one metal electrode) that is not smaller than two times a width of the at least one metal electrode at its distal end. The connection region can be formed as a flap or wing extending away from the bar-shaped sensing region of the at least one metal electrode. Thereby, landing pads for electrical pins of a mounting bracket of the measurement system can be formed.

Thereby, a reliable and robust electrical contact can be established.

The base can include one or more engagement features for aligning the dip-in-well sensor and the mounting bracket of the measurement system.

The one or more engagement features can be formed as protrusions or ridges or notches in a circumference of the base. The one or more engagement features can assist the attachment procedure of the sensor to the mounting bracket. A reliable relative alignment can be provided. This avoids relative movement of the sensor and the mounting bracket during the measurement. This reduces noise in the signals acquired from the sensor. This provides higher accuracy for the electrochemical measurement.

For example, one or more magnets may be provided, to attach the sensor to the mounting bracket of the measurement system using magnetic force. This can enable reliable and easy attachment.

As a general rule, it would be possible that the at least one metal electrode arranged on the top or bottom surface of the base and the further metal electrode arranged on the top surface of the substrate slab are made from different metals. Different manufacturing processes for depositing such metal can be used. For instance, the at least one metal electrode that is arranged on the top surface of the elongated base can be made of screen-printed silver. The further metal electrode can be made of a gold thin film. Such gold thin film can be deposited, e.g., vapor deposited or sputtered. The elongated base can be made of acrylic.

A method of manufacturing a dip-in-well sensor for electrochemical measurements includes obtaining an base. The method also includes forming at least one metal electrode on the base. The method also includes obtaining a substrate slab. The method also includes forming a further electrode on the substrate slab. The method further includes attaching the substrate slab to the base after forming the at least one metal electrode and after forming the further electrode.

In some examples, the at least one metal electrode is made of screen-printed silver. Accordingly, it would be possible to form the at least one metal electrode by screen-printing silver.

In some examples, the further electrode can be made of or at least include gold or another noble metal. The further electrode may include Platinum or Aluminum. Forming the further electrode can include depositing a thin film of such material. For instance, vapor deposition or sputtering or thermal deposition are possible options.

By separately preparing the at least one electrode on base and the further electrode on the substrate slab, degradation of the further electrode or a bio-functionalization thereof due to the process of forming the at least one metal electrode is avoided. Furthermore, more flexibility is provided in the bio-functionalization, because the at least one further electrode or the base are separated from the substrate slab at this time.

A measurement system for performing electrochemical measurements of assays in wells of a multi-well played is disclosed. The measurement system includes a base, e.g., a base plate or housing or frame.

A robotic actuator is arranged on the base and includes a moveable platform. The moveable platform can be relatively moveable with respect to a multi-well plate engaged by a sample holder arranged on the base of the measurement system. For instance, the robotic actuator may have two degrees of freedom to reposition the moveable platform in a plane parallel to the multi-well plate.

The measurement system also includes a mounting bracket. The mounting bracket is arranged on the moveable platform. I.e., the mounting bracket moves along with the moveable platform. The mounting bracket is for releasably engaging a dip-in-well sensor work. The dip-in-well sensor includes multiple electrodes. Aspects with respect to such dip-in-well sensor have been disclosed above. The dip-in-well sensor can be dipped into the multi-well plate by a respective dipping mechanism attached to the moveable platform (e.g., a linear motor). It would also be possible that the moveable platform is repositionable in three dimensions, i.e., to flexibly reposition the moveable platform with respect to different wells of the multi-well plate as well as dip the sensor into a respective well.

The multiple electrodes are for acquiring signals based on which the electrochemical measurement is performed. For instance, a current flow or potential difference between the multiple electrodes can be sensed.

The measurement system also includes an analog-to-digital converter. The analog-to-digital converter is also direction of the moveable platform. I.e., the analog-to-digital converter moves along with the moveable platform. The analog-to-digital converter is configured to electrically couple to the multiple electrodes of the dip-in-well sensor when the dip-in-well sensor is engaged by the mounting bracket and to convert analog electrical signals are obtained from the dip-in-well sensor to digital signals.

A control circuitry of the measurement system is arranged on the base of the measurement system and is configured to perform the electrochemical measurements based on digital signals.

By arranging the analog-to-digital converter on the moveable platform, the signal path between the sensor and the analog-to-digital converter is reduced if compared to a scenario in which the analog-to-digital converters arranged on the base. Thereby, the signal-to-noise ratio can be increased. Thereby, the quality of the electrochemical measurements can be increased.

The mounting bracket may be configured to releasably engage the sensor by a spring-biased clamping mechanism. For instance, a lid can be opened by the user (applying force against the spring bias) to introduce the sensor into the mounting bracket and then the lid can be closed by the spring bias to retain and fix the sensor in the mounting bracket.

Such a technique enables easy exchange of sensors. Furthermore, the sensors are fixedly retained with minimum relative movement between the sensor and the mounting bracket, thereby reducing electrical noise picked up by such movement.

To contact the multiple electrodes, the mounting bracket includes multiple contact pins, according to various examples. Also, these contact pins may be spring biased by themselves, so as to support reliable electrical contact between each pin and the electrodes.

According to examples, the mounting bracket can include, for each of the multiple electrodes, a respective pair of contact pins. Then, it is possible that the control circuitry measures a current flow in between the contact pins of each pair, to thereby determine whether the dip-in-well sensor has been properly engaged by the mounting bracket. In other words, a test mode can be activated in order to test whether the electrical contact between the sensor and the analog-to-digital converter is properly established. For instance, current variations can be monitored and if the current variations exceed a certain threshold, it can be judged that the mounting bracket only loosely engages the sensor. Alternatively or additionally, it is signal level of a current flowing in between the contact pins of each pair can be determined and based on the current amplitude it can be judged whether the mounting bracket properly engages the sensor.

Upon completing the test mode, i.e., upon determining that dip-in-well sensor has been properly engaged by the mounting bracket, the measurement mode may commence. In the measurement mode, the control circuitry is configured to perform the electrochemical measurement based on a current flow, e.g., through a single one of each pair of contact pins. For instance, the current flow in between contact pins of two different pairs can be monitored. Thereby, the amount of channels required by the ADC is reduced.

The mounting bracket can include structural guide features to align the dip-in-well sensor with multiple contact pins of the mounting bracket. Such guide features can include a notch to engage with the corresponding protrusion of the sensor. Likewise, a protrusion of the mounting bracket could engage with the corresponding notch of the sensor. The notch can run in a direction perpendicular to an offset in between the multiple contact pins. Thereby, it is ensured that the contact pins are properly placed on respective contact regions of the electrodes.

It is to be understood that the features mentioned above and those yet to be explained below may be used not only in the respective combinations indicated, but also in other combinations or in isolation without departing from the scope of the invention.

### BRIEF DESCRIPTION OF THE DRAWINGS

FIG. 1 is a top perspective view of a dip-in-well sensor 100 for electrochemical measurements according to various examples.

FIG. 2 schematically illustrates the connection of the sensor of FIG. 1 to a measurement system according to various examples.

FIG. 3 is a flowchart of a method of manufacturing a dip-in-well sensor for electrochemical measurements according to various examples.

FIG. 4 schematically illustrates details with respect to the measurement system.

FIG. 5 schematically illustrates a mounting bracket for releasably engaging a dip-in-well sensor according various examples.

FIG. 6 is a top perspective view of a dip-in-well sensor 100 for electrochemical measurements according to various examples.

FIG. 7 is a bottom perspective view of the dip-in-well sensor 100 of FIG. 6.

FIG. 8 is a perspective top view of multiple sensors being arranged in a common mounting bracket according to various examples.

FIG. 9 is a perspective bottom view of the system of FIG. 8.

FIG. 10 is a schematic top view of multiple sensors having a common base according to various examples.

FIG. 11 is a schematic bottom view of the system of FIG. 10.

### DETAILED DESCRIPTION

In the following, embodiments of the invention will be described in detail with reference to the accompanying drawings. It is to be understood that the following description of embodiments is not to be taken in a limiting sense. The scope of the invention is not intended to be limited by the embodiments described hereinafter or by the drawings, which are taken to be illustrative only.

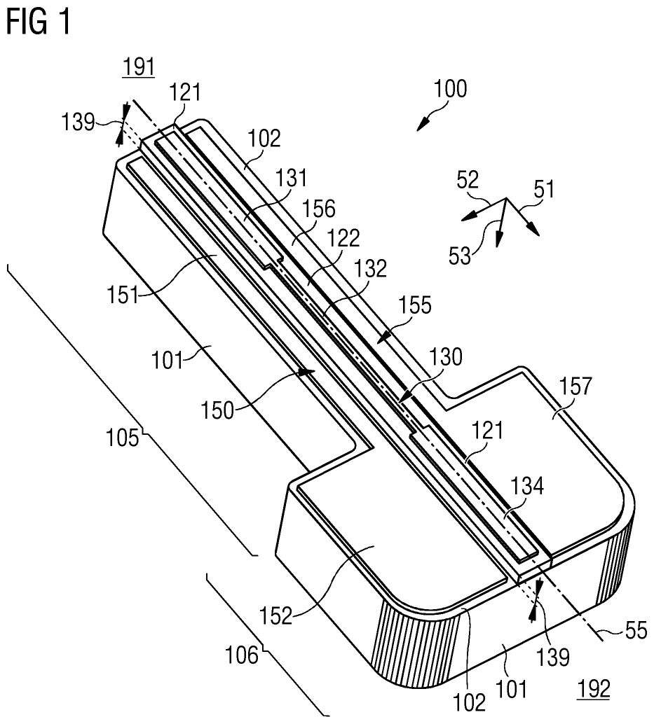

FIG. 1 is a perspective view of a dip-in-well sensor 100 according to various examples. The sensor 100 has generally an elongated shape, i.e., extends along a longitudinal direction 51. A respective longitudinal center axis 55 (arranged in the middle of the sensor 100 along a width direction 52) of the sensor 100 extending between a distal end 191 and a proximal end 192 is illustrated (dashed dotted line).

The sensor 100 includes an elongated base 101 that extends between the distal end 191 and the proximal end 192.

The base 101 has a top surface 102 and a bottom surface (obstructed from view in FIG. 1). The top surface 102 is plane and extends in a plane defined by the longitudinal direction 51 and a width direction 52.

The base 101 includes a narrow sensing region 105 (for dipping into a well) at the distal end 191 and a wider connection region 106 for enabling electrical contact between a measurement system and multiple electrodes 150, 155, 130 of the sensor 100.

The metal electrodes 150, 155 are arranged on the top surface 102 of the base 101 and extend between the distal end 191 and the proximal end 192. For instance, the metal electrode 150, 155 can be made of silver (specifically, silver paste) by a screen-printing process.

The metal electrode 130 is arranged on a top surface 122 of an elongated substrate slab 121 that is attached, at its bottom surface (obstructed from view in FIG. 1), to the top surface 102 of the base 101. The elongated substrate slab 121 extends along the metal electrodes 150, 155 between the distal end 191 and the proximal end 192.

Arranged on the top surface 122 of the elongated substrate slab 121 is the electrode 130, e.g., a thin metal film possibly including gold. Such thin film can be defined by lithography, e.g., by defining a lithography mask and then depositing the thin film, or by first forming the thin film and then etching using a lithography mask.

By attaching the substrate slab 121 onto the top surface 102 of the base 101, the elongated substrate slab 121 protrudes from the top surface 102 of the elongated base 101 as well as from the metal electrodes 150, 155. The respective offset 139 is marked in FIG. 1.

This offset 139—extending in a thickness direction 53, perpendicular to the longitudinal direction 51 and the width direction 52—facilitates the alignment of the sensor 100 in a mounting bracket of the measurement system, by providing guidance for relative positioning along the width direction 52.

As illustrated in FIG. 1, the arrangement of the electrodes 150, 155, 130 is mirror-symmetrical about the center axis 55. The electrode 130 is arranged in-between the electrodes 150, 155 along the width direction 52.

In particular, a distance between a sensing region 151 of the electrode 150 and a sensing region 131 of the electrode 130 equals a distance in-between the sensing region 131 of the electrode 130 and a sensing region 156 of the electrode 155. Accordingly, it is equally possible to implement the electrochemical measurement by determining a current or a voltage in-between the pair of electrodes 130, 150 or between the pair of electrodes 155, 130.

The sensing region 131 of the electrode 130 can be bio-functionalized (not shown).

Adjacent to the sensing region 131 is a connection region 132 that is tapered in the width direction 52 if compared to the sensing region 131, as well as with respect to a connection region 134 to engage with one or more electrical pins of a mounting bracket of a measurement system, thereby establishing electrical contact. By providing the connection region 132 with a smaller width than the sensing region 131, variable fill levels of the assay resulting in different degrees of coverage of the connection region 132 have a smaller impact onto the measurement signal if compared to the scenario in which the connection region 132 would have the same width as the sensing region 131 that is bio-functionalized. The reduced width of the connection region 132 renders the overall electrochemical measurement more accurate.

Also illustrated in FIG. 1 is the connection region 152 (may also be termed “contact region”) of the electrode 150 having a wider width (along the width direction 52) than the sensing region 151 to be dipped into the assay. Likewise, the width of the connection region 157 of the electrode 155 is larger than the width of the sensing region 156 of the electrode 155. The width of the connection regions 152, 157 of the electrodes 150, 155 can be generally not smaller than three times the width of the electrodes 150, 155 at the distal end 191. This facilitates, both, reliable electrical contact to pins of the measurement system at the connection regions 152, 157; as well as the ability to dip the sensor 100 into a narrow wells.

The shape of the elongated base 101 having the wide connection region 106 and the narrow sensing region 105 facilitates aligning and mounting the sensor 100 in a mounting bracket of a measurement system. In particular, the footprint of the connection region 106 of the base 101 can be aligned in a corresponding indentation of the mounting bracket, which is facilitated by the edges of the connection region 106. As a general rule, the elongated base 101 can include one or more engagement features for aligning the sensor 100 in the mounting bracket, e.g., ridges, edges, etc. formed in a circumference of the base 101. It would also be possible that the sensor 100 includes one or more magnets to magnetically attach the sensor 100 to the mounting bracket. For instance, the magnets can be attached to the base 101 at the connection region 106 (not shown in FIG. 1).

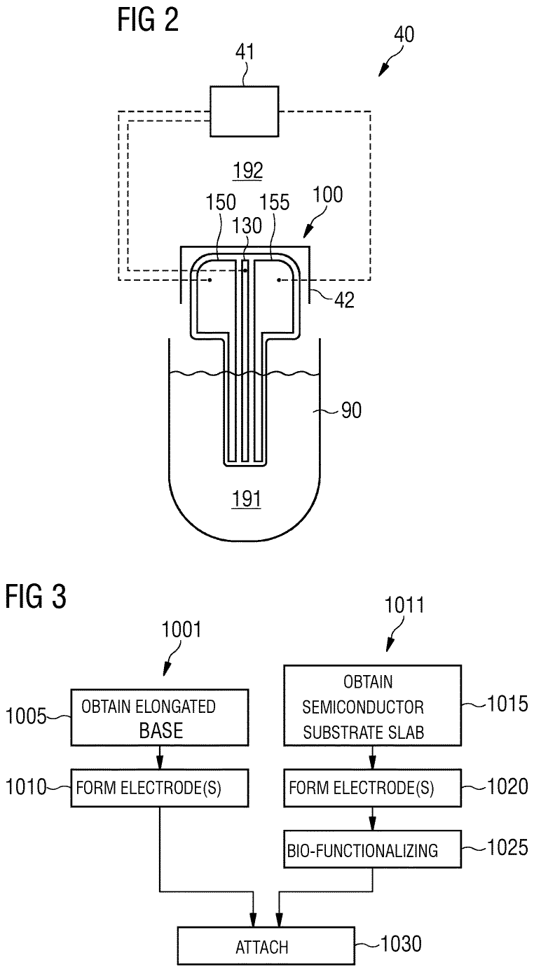

FIG. 2 schematically illustrates the sensor 100 being aligned and attached to a mounting bracket 42 of a measurement system 40 so that an electrical connection between the connection regions of each of the electrodes 150, 155, 130 and a control circuitry 41 of the measurement system 40 is established. It is illustrated that the sensor 100 is submerged into the in an assay 90 at the distal end 91.

FIG. 3 is a flowchart illustrating a method of manufacturing a dip-in-well sensor for electrochemical measurements. For instance, the sensor 100 according to FIG. 1 may be manufactured using the method of FIG. 3. FIG. 3 illustrates a manufacturing method providing a multi-part sensor with separate processing strings 1001 and 1011.

In the processing string 1001 an elongated base is obtained at box 1005, e.g., made of acrylic. Then, one or more electrodes are formed on that base at box 1010. This may include applying relatively high temperatures, e.g., around 150 °C to 200 °C. For instance, multiple electrodes can be screen-printed. Silver electrodes can be formed. Silver screen printing is a technique used to apply metallic silver ink onto a variety of surfaces, such as paper, fabric, or plastic. It involves creating a stencil of the desired design on a fine mesh screen, with the areas to be printed left open. The screen is then placed on the surface to be printed, and silver ink is pushed through the open areas using a squeegee. The ink is transferred onto the surface, creating the desired metallic silver design. Finally, the printed material is dried or cured to ensure the ink adheres properly. Typical curing temperatures range from around 150 to 175 °C.

Such temperatures have the potential to damage bio-functionalized surfaces.

Accordingly, the second manufacturing string 1001 is separated from the first manufacturing string 1001.

At box 1015, a substrate slab is obtained. For instance, a chip can be obtained by cutting a wafer. Typical dimensions of the substrate slab are as follows: thickness: 250 μm-500 μm; length along a longitudinal direction of the elongated substrate slab: 1 cm-5 cm; width perpendicular to the thickness and longitudinal direction: 1 mm-1 cm.

Then, at box 1020, electrodes are formed on the elongated substrate slab using a lithography mask. For instance, the lithography mask can be first formed on the elongated substrate slab and then metal for the electrodes, can be deposited. For instance, chemical or physical vapor deposition can be used. Subsequently, the mask can be removed. Alternatively, first a continuous gold film can be deposited and subsequently, the mask can be formed on the gold film (an inverse mask if compared to the first scenario), and subsequently excess gold can be removed by etching, e.g., wet etching or dry etching.

At box 1025, at least a part of the one or more electrodes formed at box 1020 can be bio-functionalized. There are various methods available for bio-functionalizing the electrodes such as forming self-assembled monolayers. Here, sulfur in an alkanethiol adheres to the electrode surface while the other end of the molecule can be functionalized with various chemical groups. In another option, biomolecules can be directly adsorbed onto the surface, using the affinity between metal and certain functional chemical groups. In yet a further option, a layer-by-layer-assembly is possible where oppositely charged polyelectrolytes, proteins, or nanoparticles form a multi-layer structure. In yet a further scenario, biomolecules can be covalently bonded to the surface of electrodes via bio conjugation. A reactive group of the biomolecule can be used or a linker molecule can be relied upon that links the electrode surface and the biomolecules. Another option is electrochemical grafting where certain organic molecules can be grafted onto the surface of the electrodes using electrochemical process. In any of such scenarios, the bio-functionalized surface of the electrode can be sensitive to the temperatures required to form the electrodes at box 1010; which makes it convenient to separate the processing strings 1001 and 1011.

Finally, at box 1030, the substrate slab carrying the one or more electrodes formed at box 1020 is attached to the base carrying the one or more electrodes formed at box 1010. For instance, an adhesive connection can be formed.

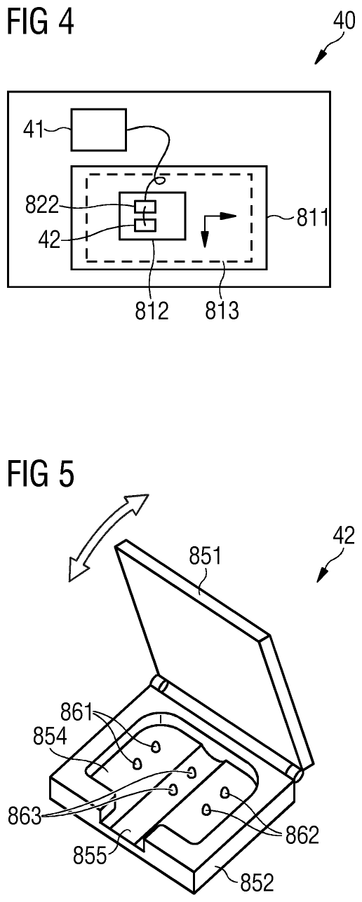

FIG. 4 schematically illustrates details with respect to the measurement system 40. The measurement system 40 includes the control circuitry 41. The control circuitry 41 can be implemented by a general-purpose central processing unit (CPU), a field-programmable gate array (FPGA), and/or an application-specific integrated circuit (ASIC). The control circuitry 41 can include a memory that stores program code for being executed by the CPU, the FPGA or the ASIC. The control circuitry 41 is configured to perform an electrochemical measurement based on digital signals that are obtained from an analog-to-digital converter (ADC) 822. The digital signal obtained from the ADC 822 is indicative of a current flow or potential difference between electrodes of a dip-in-well sensor such as the sensor 100 discussed above in connection with FIG. 1.

The control circuitry 41 is arranged on a fixed base 810 of the measurement system 40. The ADC 822 is arranged on a moveable platform 812 of a robotic actuator 811. The moveable platform 812 can move with respect to the fixed base 810 by means of the robotic actuator 811. The robotic actuator 811 is configured to reposition the moveable platform 812 within a certain measurement area 813. For instance, such repositioning can have two degrees of freedom, as indicated in FIG. 4 using the arrows. For instance, two motors can be provided and coupled via a belting system with the moveable platform 812. It would also be possible to use a robotic arm having more than two degrees of freedom.

The ADC 822 converts analog signals obtained from the sensor engaged by the mounting bracket 42 also arranged on the moveable platform 812.

By provisioning the ADC 822 of the moveable platform 812, the cable length between the sensor engaged by the mounting bracket 42 and the ADC 822 can be dimensioned comparably short. Accordingly, the signal-to-noise ratio of the analog signal converted by the ADC 822 is comparatively high. This increases the accuracy of the electrochemical measurements performed by the control circuitry 41. It has been found that such placement of the ADC 822 in close proximity to the sensors helpful for implementing the electrochemical measurements at high accuracy, because often times the currents measured are in the order of nanoamps.

FIG. 5 schematically illustrates a possible implementation of the mounting bracket 42. In the example of FIG. 5, the mounting bracket 42 is shaped to releasably engage the sensor 100.

For this, a first notch or recess 854 is provided that is shaped to engage the connection region 106 of the base 101 of the sensor 100; also, a second notch or recess 855 is provided that has a shape matched to the protruding substrate slab 121. More generally, the mounting bracket can include multiple engagement or guide features to align the electrodes of the sensor with multiple contact pins 861, 862, 863 of the mounting bracket 42.

In the scenario of FIG. 5, the mounting bracket 42 includes a lid 851 that is configured to releasably engage the sensor. The lid 851 may be spring-biased. More generally, other types of clamping mechanisms would be conceivable.

It is also possible that the multiple contact pins 861-863 are spring biased. I.e., the contact pins 861-863 can be pressed against the surfaces of the electrodes by a respective spring-biased force.

As illustrated in FIG. 5, the mounting bracket 42 includes three pairs of electrical contact pins 861-863. There is one pair of electrical contact pins 861-863 per electrode 130, 150, 155. I.e., the connection regions 134, 152, 157 of each of the electrodes 130, 150, 155 is contacted by two respective contact pins 861-863. For instance, referring to FIG. 1, the connection region 152 can be contacted by both contact pins 861; the connection region 134 can be contacted by both electrical contact pins 863; and the connection region 157 can be contacted by both contact pins 862. The control circuitry 41 of the measurement system 40 can then, in a test mode, measure a current flow in between the contact pins of each pair, e.g., between the contact pins 861 of the respective pair, to determine whether the sensor has been properly engaged by the mounting bracket 42. For instance, only in a sufficiently large current flows measured between the contact pins of each of the three pairs, it can be judged that the sensor has been properly engaged and, subsequently, the electrochemical measurement may be performed. Alternatively or additionally, a current variation over time may be monitored. The electrochemical measurement can then be based on a current flow through a single contact pin of each pair. For instance, the electrochemical measurement can be based on a current flow from one of the electrical contact pins 863 to one of the electrical contact pins 861. By only using a single contact pin per electrode for the actual electrochemical measurement, the amount of channels required that the ADC is reduced.

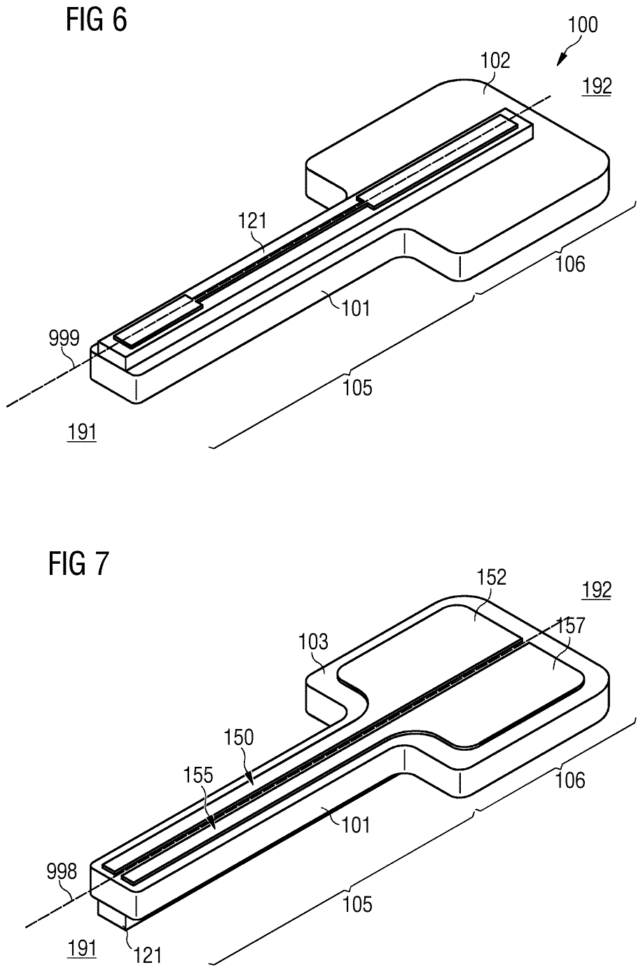

Above, scenarios have been discussed in which the metal electrodes 150, 155 are arranged on the top surface 102 of the elongated base 101. This is only one example and a further example is illustrated in FIG. 6 and FIG. 7. FIG. 6 is a top perspective view of a further implementation of the sensor 100. Like the implementation in FIG. 1, the elongated substrate slab 121 is attached to the top surface 102 of the elongated base 101. In this scenario, the metal electrodes 150, 155 are arranged on the bottom surface 103 of the elongated base 101, as is apparent from the bottom perspective view of FIG. 7. As is apparent from FIG. 6 and FIG. 7, the metal electrodes 150, 155 are arranged symmetrically with respect to a center axis 998 that is parallel to the center axis 999 of the electrode 130. Both, the center axis 998 as well as the center axis 999 and the center axis 55 of the sensor (cf. FIG. 1) are aligned with each other and in one plane.

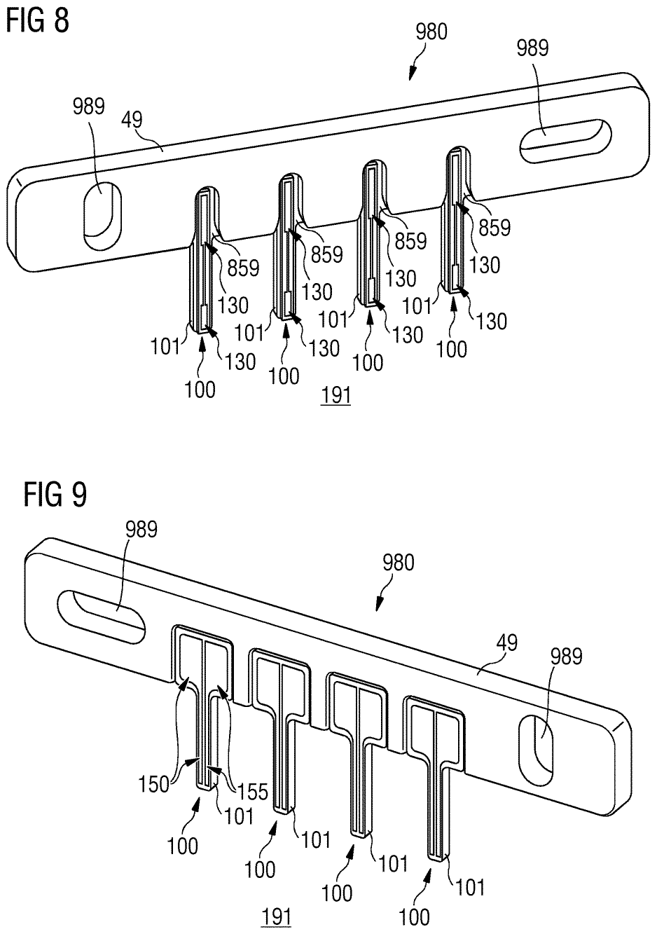

FIG. 8 is a top perspective view of a system 980 including multiple sensors 100. The multiple sensors 100—here four sensors, but a larger or smaller count would be generally possible—are arranged in a row. In the illustration of FIG. 8, the sensors 100 are each configured in accordance with the variant of FIG. 6 and FIG. 7; however, as a general rule, the sensors could also be configured differently, e.g., in accordance with the variant of FIG. 1 (i.e., all electrodes 130, 150, 155 arranged at the same side of the sensor 100).

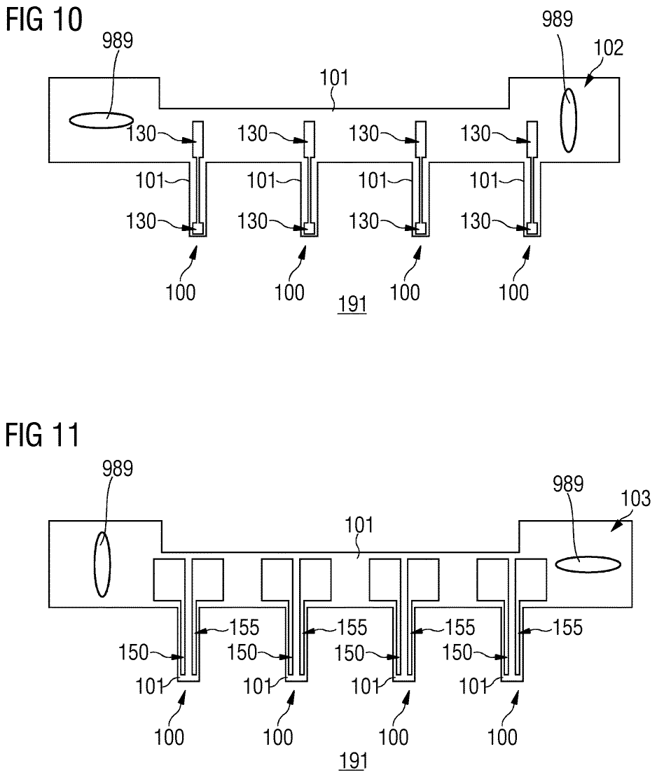

The system 980 also includes a mounting structure 49 that can engage with a clamp (cf. FIG. 5: bracket 42) for mounting the system. The bases 101 of the sensors 100 are attached to the mounting structure 49, e.g., by adhesive. The mounting structure 49 includes a through hole that enables contacting the connection region 134. This is visible in FIG. 9 which is the bottom perspective view corresponding to the top perspective view of FIG. 8.

Where multiple sensors 100 are mounted next to each other, as illustrated in FIG. 8 and FIG. 9, it is possible that a single base 101 is used for the multiple sensors 100. This is illustrated in the schematic top view of FIG. 10 and the schematic bottom view of FIG. 11. Such a scenario can also be applied for configurations in which the metal electrodes 150, 155 are attached to the top surface 102 of the base, e.g., as discussed in connection with FIG. 1. Such configuration facilitates the use of a common mounting bracket (not shown in FIG. 10 and FIG. 11) that engages the single base 101 at respective engagement features 989 at the outer ends (through holes). Such implementation of the system 980 using a single, integral base 101 has the following advantages: easier manual handling avoiding the need to individually handle each small sensor 100. In the implementations of FIGS. 8-11, it is possible to have larger engagement features 989 at both ends of the row of sensors 100, enabling simple attachment.

Although the invention has been shown and described with respect to certain preferred embodiments, equivalents and modifications will occur to others skilled in the art upon the reading and understanding of the specification. The present invention includes all such equivalents and modifications and is limited only by the scope of the appended claims.

For illustration, the surfaces of the substrate slab as well as the surfaces of the base have been described as ‘top surface’ and “bottom surface'. This should not be understood as limiting or absolute terms: the top and bottom surfaces are arranged on opposing sides of the respective element.

1. A dip-in-well sensor for electrochemical measurements by being dipped into an assay at a distal end, the dip-in-well sensor comprising:
a base,
at least one metal electrode arranged on a first surface or a second surface of the base and extending between the distal end and a proximal end of the dip-in-well sensor,
a substrate slab attached, at its second surface, to the first surface of the base and extending along the at least one metal electrode between the distal end and the proximal end, and
a further metal electrode arranged on a first surface of the substrate slab and extending between the distal end and the proximal end.

2. The dip-in-well sensor of claim 1,
wherein a sensing region of the further metal electrode arranged at the distal end is bio-functionalized,
wherein a connection region of the further metal electrode extending from the sensing region towards the proximal end has a smaller width than the sensing region.

3. The dip-in-well sensor of claim 1,
wherein the substrate slab protrudes from the first surface of the base.

4. The dip-in-well sensor of claim 1,
wherein the at least one metal electrode comprises a first metal electrode and a second metal electrode.

5. The dip-in-well sensor of claim 4,
wherein the first metal electrode and the second metal electrode are symmetrically arranged with respect to a center axis that is parallel with a center axis of the further metal electrode.

6. The dip-in-well sensor of claim 1,
wherein the base comprises a narrow dipping region and a wide connection region for enabling electrical contact between the at least one metal electrode and a measurement system, as well as between the further metal electrode and the measurement system.

7. The dip-in-well sensor of claim 6,
wherein the at least one metal electrode comprises a connection region 152 arranged in the wide connection region of the base and having a width perpendicular to a longitudinal direction of the at least one metal electrode that is not smaller than three times a width of the at least one metal electrode at the distal end.

8. The dip-in-well sensor of claim 1,
wherein the base comprises one or more engagement features for aligning the dip-in-well sensor in a mounting bracket of a measurement system.

9. The dip-in-well sensor of claim 1, further comprising:
one or more magnets to attach the dip-in-well sensor to a mounting bracket of a measurement system.

10. A system comprising multiple dip-in-well sensors according to claim 1.

11. The system of claim 10,
wherein the bases of the multiple dip-in-well sensors are integrally formed.

12. The system of claim 10,
wherein the multiple dip-in-well sensors are arranged in a row,
wherein the system further comprises:
multiple engagement features arranged at both ends of the row for aligning the dip-in-well sensor in a mounting bracket of a measurement system.

13. A method of manufacturing a dip-in-well sensor for electrochemical measurements, the method comprising:
obtaining a base,
forming at least one metal electrode on the base,
obtaining a substrate slab,
forming a further electrode on the substrate slab,
after forming the at least one metal electrode and the further electrode, attaching the substrate slab to the base.

14. The method of claim 13, further comprising:
bio-functionalizing at least a part of the further electrode,
wherein the substrate slab is attached to the base after bio-functionalizing the at least part of the further electrode.

15. The method of claim 13,
wherein the manufactured dip-in-well sensor is a dip-in-well sensor.
